(19) United States
(12) Patent Application Publication
(10) Pub. No.: US 2021/0322329 A1
(43) Pub. Date: Oct. 21, 2021

(54) NANOSTRUCTURED NANOPARTICLES COMPRISING ONE OR MORE ACTIVE INGREDIENTS FOR THE TREATMENT OF DISEASES CAUSED BY TRYPANOSOMES AND FOR THE TREATMENT OF TUMOURS OF NEURAL ORIGIN, COMPOSITIONS COMPRISING SAME, A PREPARATION METHOD AND THERAPEUTIC USE THEREOF

(71) Applicant: GADOR LIMITADA, Santiago (CL)

(72) Inventors: Valentina Carricarte, Santiago (CL); Emilio Roldan, Santiago (CL)

(21) Appl. No.: 17/270,342

(22) PCT Filed: Aug. 21, 2018
(86) PCT No.: PCT/CL2018/050067

§ 371 Date: Feb. 22, 2021

(51) Int. Cl. A61K 9/51; A61K 31/541; A61P 33/02

(52) U.S. Cl. CPC A61K 9/5123; A61P 33/02; A61K 31/541; A61K 9/5192

(57) ABSTRACT

Pharmaceutical composition of antineoplastic and anti-tumor action, and method of obtaining it, wherein said composition comprises structured lipid nanoparticles that are between 0.1 nm and 100 nm in diameter, spheroidal in shape and contain particles of one or more active substances for the treatment of diseases produced by tripanosomes and for the treatment of tumors of neural origin, and at least one coupling of two fatty acids consisting of a saturated fatty acid or semi-solid lipid at the temperature between 19° C. and 21° C. and an unsaturated or liquid fatty acid at the temperature between 19° C. and 21° C. and in turn the method of obtaining finally contains the nanoparticles of one or more active substances for trypanosomiasis in colloidal suspension, the Z potential ranges from −30 to +30, preferably +8, the particles measured by Z-sizer and ultramicroscopy have from 0.1 nanometers to 100 nanometers, preferably 1 nanometer in diameter and spheroidal shape.

### 1) TECHNICAL FIELD OF INVENTION

The present invention refers to a “Pharmaceutical composition of antineoplastic and antitumor action and method of obtaining it”, applied in the field of diseases produced by trypanomas, preferably for the treatment of Chagas Mazza disease and for the treatment of tumors of neural origin, the central nervous system such as astrocytoma, retinoblastoma and neuroblastoma.

### 2) TECHNICAL STATUS AND PROBLEMS DETECTED

Chagas disease is currently endemic in Latin America and is a major public health problem throughout the region. It is estimated that this disease affects approximately 15 to 20 million people, from Southern California to Argentina and that more than 100 million individuals would be exposed to the risk of infection. Thus, it is estimated that between 2 and 3 percent of Latin America's population would be infected. Chagas morbidity and mortality are ten times higher than those exhibited by other parasititarian diseases in the subcontinent [1, 2, 3, 4]. The causal agent of Chagas disease is the flagellated protozoa Trypanosoma cruzi, which is transmitted to humans through the bite of vinchuca (Triatoma infestans) and other hematophageal insects in the Family Reduviidae^{[5]}. In most cases (>80%), human infection is related to insect faeces, blood transfusion or organ transplantation (15%)^{[6]}. The discovery of an indigenous transmission makes Chagas a potentially emerging disease in the United States^{[7].} In addition, Latin American emigration has brought this disease to developed countries in Europe and Oceania^{[8]}.

The biological cycle of the parasite consists mainly of three fundamental cellular forms:

(a) trypomastigotes, which are found in mammalian blood, spreading the infestation transmitted from the hematophageal intestine;
b) epimastigotes, which is the proliferative form present in the insect's intestine; And
c) the amastigotes, which multiply by binary fission within mammalian host cells, producing their lysis and releasing new trypomastigotes into the bloodstream. (See Functional effectiveness of nifurtimox nanoparticles in the in vitro treatment of chagas disease). In this way, they can invade any nucleated cell and begin a new reproductive cycle^{[9]}.

The infection takes place in three phases: acute, indeterminate and chronic. The acute phase of the disease is characterized by high parasitemia, consisting of trypomastigotes. After a period ranging from a few weeks to several months, the infection is usually well controlled by the host's immune response. However, parasites follow their cycle in and out of mammalian cells and appear in the bloodstream only occasionally, making it difficult to detect. Symptoms are often not long apparent (perhaps decades), so this period is referred to as an indeterminate phase. About 40% of infected patients end up developing the chronic phase of the disease, where intracellular kneadings cause irreversible structural damage to the esophagus, colon and heart^{[2]}.

The first-choice drugs authorized for the treatment of Chagas disease are nifurtimox (3-methyl-4-(5′-nitrofurfuriliden-amino)-tetrahydro-4H-1,4-thiazine-1,1-dioxide, Bayer 2502) and benznidazole (2-nitroimidazole (N-bencil-2-nitroimidazole) acetamide, RO 7-1051). It has been suggested in various studies that etanidazole, derived from nitroimidazole, eflornitine, melarsoprol, pentamidine, surimin, fexinidazole, could also be used. These two drugs (nifurtimox and benznidazole) have been used for this purpose for decades [10] and, to date, have not been able to be replaced, even though both exhibit serious side effects and have low efficacy^{[11-15].} Both drugs are effective in the acute phase, chronic infections of children up to 12 years of age and in congenital infections^{[2]}, but have minimal or no activity during the chronic phase of the disease. In In vitro, nifurtimox and benznidazole manage to kill the intracellular form of the parasite (amastigotes). Circulating parasites (trypomastigotes) are also destroyed by these live agents, but no benefit is verified when these drugs are used in adult humans during the indeterminate and chronic phases of the disease^{[19].} After intestinal absorption, these drugs easily reach tissues, but high doses are required to remove circulating parasites. Thus, it has been proposed that an increase in the arrival of drugs into affected tissues (reservoirs) could improve therapeutic efficacy^{[20]}.

To date, no vaccines or effective treatments are available on the market for adults with Chagas disease in its chronic phase^{[16]}. This is mainly because Chagas belongs to the group of so-called “forgotten” diseases, which are endemic in the low-income populations of developing regions of Africa, Asia and the Americas^{[17]}. The lack of better pharmacotherapeutics is due to the absence of effective public policies in countries suffering from endeemia^{[18]}. Private pharmaceutical companies have not shown interest either.

In 2007, the World Health Assembly (WHA) proclaimed a child's right to access safe, effective and proven medicines. But existing medicines, nifurtimox and benznidazole, exist only in solid pharmaceutical forms. High doses and complex administration schedules affect the lifestyle of young patients and decreased adherence levels dramatically restrict treatment success^{[21]}.

Children can be cured through liquid formulations of nifurtimox or benznidazole, but these are not commercially available. This is why liquid forms are called “orphan drugs”. In this context, the office processing of the original solid pharmaceutical forms^{[22]} becomes the only alternative for the treatment of paediatric patients of the disease. However, due to the extreme insolubility of the active substances, the erratic bioavailability of their suspensions and the serious lack of resources in countries with fragile health systems and limited infrastructure, the quality, safety and efficacy of these extemporaneous formulations are doubtful^{[23-25]}.

For these reasons, there is an urgent need for new and effective anti-chagassic products. Currently, there are mainly two possible strategies for carrying out programmes to produce new medicines for the treatment of infected patients: (i) encourage the search for new drug candidates or (ii) develop improved drugs for the administration of drugs already approved and available on the market^{[26]}.

In this sense, the design and development of new antiparasitic drug release systems that are safe, effective and efficient is indispensable. In particular, new systems are needed for the release of nifurtimox, benznidazole, and the rest of those mentioned herein to reduce the dose to be administered, improving its absorption. There is also an unmet need to develop liquid formulations of nifurtimox and benznidazole, in particular pharmaceutical forms usable in pediatrics.

The preparation of conventional and stable pharmaceutical forms based on nifurtimox or benznidazole is complex, due to its physical-chemical properties and, in particular, its low solubility in water. In recent years nano-sized objects (nanoparticles, 0.1 nanometers to 1000 nanometers in diameter, preferably 1 nanometer to 500 nanometers) have been proposed as hard-to-use drug vehicles in terms of bioavailability, solubility, pharmacokinetics, etc. Liposomes, known for several decades, contain an interior cavity suitable for transport. However, liposomes loaded with benznidazole showed no parasiticide activity. In addition, unilamellary liposomes suffer from fragility, so they release the drug early, while multi-lamp liposomes fail in the “delivery” of the active substance, as they are excessively resistant and do not release the drug. Other attempts to use different nano objects have collided, until the filing of this patent, with the very toxicity of the vehicles chosen^{[37]}.

Certain types of nano objects, solid lipid nanoparticles (SLNs), have the following advantages when used for the controlled distribution of drugs: a high tissue tolerance, few regulatory requirements if physiologically acceptable lipids are used, the ability to transport lipophilic and hydrophilic drugs, protection against chemical degradation of the active substances, the possibility of setting the target and low cost of production. However, they show some intrinsic limitations, such as its low carrying capacity and a tendency to expel the drug during the storage period^{[33-35]}.

A variety of SLNs, called structured lipid nanoparticles (NLCs, mentioned above), have shown sharing the favorable characteristics mentioned above, while not possessing the above limitations.

In the same conceptual order, there are other forms of trypanosomiasis, for example, African Trypanosomiasis, also called sleep disease, which is caused by Tripanosoma brucei gambiensis. This parasite is also sensitive to nifurtimox and therefore the improvement of the pharmaceutical/pharmacological properties of nifurtimox must benefit the therapeutic schemes for this disease.

It is also known that nifurtimox has been used to mitigate the growth of the most common extraranial solid tumor in children, Neuroblastoma, which has very poor prognosis. The mechanism of action of nifurtimox on neuroblastoma in vitro tumor cells has been shown to be the acceleration of apoptosis^{[38-39]}. This mechanism of action on certain host tumor cells would allow its use in non-parasitial diseases. Inventors have tested, with very good results, nifurtimox nanoparticles in in vitro astrocytoma cultures and, surprisingly in Retinoblastoma (see In vitro Effectiveness of Nifurtimox Nanoparticles on Tumor Cell Apoptosis of the Central Nervous System). In the case of Astrocytoma in spherical and adherent MSP12 cell lines, Spherical and adherent MGG8 and GL26 NT (rat) and, to this day unpublished, in the case of Retinoblastoma in the Y79 cell line. Therefore, the pharmaceutical-pharmacological application of nifurtimox nanoparticles will benefit the usual therapeutic schemes for these diseases and/or enable indications in tumor variants that do not now have rational treatments.

### 3) SOLUTION PROVIDED

It is known that there are various types of particles used in the administration of medicines. Depending on their size, they can be classified into microparticles and nanoparticles.

The inventors of the present have developed novel structured lipid nanoparticles (NLCs) comprising particles of one or more active substances for the treatment of trypanosomiasis with, at least one coupling of two fatty acids consisting of a saturated fatty acid or a semi-acid lipid at the temperature between 19 degrees Celsius and 21 degrees Celsius, preferably at 20 degrees Celsius; and a liquid unsaturated fatty acid or liquid lipid at the temperature between 19 degrees Celsius and 21 degrees Celsius, preferably at 20 degrees Celsius; where these nanoparticles have an average effective size of between 0.1 nm and 100 nm, preferably from 1 nanometer to 10 nanometers, particularly 1 nm, where saturated fatty acid or semi-acid lipid is selected between palmitic acid or stebaric acid, and unsaturated fatty acid or liquid lipid is selected from oleic acid, arachydonic acid, palmitoleic acid, alpha-linoleic acid, or gamma-linoleic acid. In order to improve the aqueous suspensibility of therapeutically active substances, decrease their toxicity, increase their effectiveness, and prolong the stability of pharmaceutical forms prepared with them, the inventors of the present have investigated the encapsulation of drugs within structured lipid nanoparticles (NLCs)^{[27-32]}.

As well, inventors have developed a pharmaceutical composition that includes novel nano objects, useful in the preparation of stable liquid dosing forms of active compounds against trypanosomiasis, in particular nifurtimox and benznidazole, especially intended for administration to paediatric patients.

From the highly ordered structure presented at room temperature by solid lipids such as steanic acid and palmitic acid, the incorporation of liquid lipids into the crystals of such solid lipids leads to a massive disturbance of the organization of them. The resulting matrix of lipid particles shows large imperfections in the crystalline network and leaves enough room to accommodate molecules of the active substance, which improves the drug load capacity by the structured nano vehicle^{[32].} NLCs do not have the limitations associated with SLNs, such as a small load capacity and expulsion of the drug during storage.

In conclusion, a pharmaceutical composition has been developed with nanostructured nanoparticles (NLCs, Novel Nano Lipidic Carriers comprising particles of one or more active substances for the treatment of trypanosomiasis and at least a coupling of two fatty acids consisting of a saturated fatty acid or a semi-solid lipid at the temperature between 19 degrees Celsius and 21 degrees Celsius, preferably at 20 degrees Celsius; and a liquid monounsaturated fatty acid or liquid lipid, at the temperature between 19 degrees Celsius and 21 degrees Celsius, preferably at 20 degrees Celsius; where these nanoparticles have an average effective size of between 0.1 nm and 100 nm, preferably from 1 nanometer to 10 nanometers, where saturated fatty acid or semi-acid lipid is selected between stebaric acid and palmitic acid, and unsaturated fatty acid or liquid lipid is selected from oleic acid, arachydonic acid, palmitoleic acid, alpha-linoleic acid, or gamma-linoleic acid. Así, these novel nano objects, useful in the preparation of stable liquid dosing forms of active compounds against trypanosomiasis, in particular nifurtimox and benznidazole, especially intended for administration to paediatric patients (acute intervention) and adult patients (chronic intervention).

In addition, NLC carriers of selected active substances have been developed, in different pharmaceutical forms, of antineoplastic and anti-tumor action, especially effective for tumors of neural origin, preferably Retinoblastoma and Astrocytoma, both in vitro and in vivo.

The use of nanoparticles as a drug carrier began with liposomes, which were developed by A. Bangham et al. in 1964. Its use as a micrometric drug carrier was tested by G Georgiadis et al. in 1971. The first nanometric tests are by the same author in 1972. Structured lipid nanoparticles (NLCs) were developed by Kawashima et al. In 1998, using the method of diffusion of solvents. NLCs prepared based on the method of diffusion of solvents with oleic and stesic acids were developed by Wu et al. in 2004. The idea of using nanoparticles as carriers of anti-pessic drugs (liposomes loaded with benznidazole) was published by E Romero et al. in 2004. None of this could be patented. A comparative table of the methods used by Hu et al is below. (2005) and the inventors of the present invention to prepare NLC through the solvent diffusion technique:

TABLE 1
The similarities and technical and result differences
can be seen in this table. The volumes of water, acetone and
alcohol, and the weight of fatty acids used are the same as well
as the working temperature. The technical differences lie in the
pH used at the time of precipitation and the nanoparticle
separation procedure.

| Author | Hu et al. | CIN-Nanof |
|---|---|---|
| Technical | Solvent | Solvent diffusion |
| | diffusion | |
| Particles | NLC and SLN | Nlc |
| Drug (API) | Clobetasol | Nifurtimox |
| Preparation of empty | Yes | No |
| nanoparticles | | |
| OA/SA ratio | Oleic/Steeric | Linoleic/Palmitic |
| | 30/70 | 30/70 |
| Approximate size of | 150 to 300 nm | 1 nm |
| the nanoparticles | | |
| obtained. | | |
| Solvents for | acetone/ethanol | acetone/ethanol |
| ingredients | | |
| Diffuser | distilled water | distilled water |
| Approximate | 70° C. | 70° C. |
| temperature during | | |
| preparation | | |
| Separation (1st part) | precipitation at | precipitation at |
| | pH 1.2 | pH 2.0 |
| Separation (2nd part) | centrifuged at | paper filtering |
| | 25,000 rpm | |
| Location of | in the spin | in the overnatant |
| nanoparticles | | |
| % API load | 3.3 to 3.6% p/p | 3.5 to 4.7% p/p |
| Built-in % API | 50 to 70% | 96-98% |
| Suspensibility in | Medium | very high (178 |
| distilled water | | mg/ml) |
| Z-potential in mV | −50 | +8 |

A big difference is that they according to Hu et al., would be found in the precipitate obtained by centrifugation, and instead in our technique are found in the overnatant, where they can be visually detected by their intense yellow color typical of nifurtimox that they have incorporated, in colloidal suspension, in a clear liquid. If we copied Hu's technique, we would not find any nanoparticles or nifurtimox in the precipitate obtained by centrifugation, proof that we actually perform. We also confirm this experimentally by identification and titration of the precipitate we obtained by filtration. Both precipitates are practically made up of fatty acids, with no traces of nifurtimox.

Some of the advantages of NLCs are observed in the last lines: the capture of the drug in Hu's preparation is quite good, up to 70%, but in the preparation CIN-Nanof rises to almost all nifurtimox (this solves the problem of removing from the liquid the drug not incorporated in the NLCs). The high suspensibility of the new NLCs is also very interesting, considering that it serves to pharmacotechnically solve the problem of almost total insolubility of nifurtimox-drug in water when making pharmaceutical preparations.

TABLE 2
Effects of pH on 10 mg/100 ml suspension. The wide pH
range (from 2.5 to 9) in this table shows that the NLC suspension
prepared by the CIN-Nanof is macroscopically stable.

| Ph | State |
|---|---|
| 1,7 | flocula |
| 2 | Turbidity |
| 2,5 | Opalescence |
| 3 | Transparency |
| 4 | Transparency |
| 5 | Transparency |
| 6 | Transparency |
| 7 | Transparency |
| 8 | Transparency |
| 9 | Opalescence |
| 9,5 | Turbidity |
| 10 | Flocula |

### Granulometric Measurement of NLCs

The results of the measurement of the average size of the NLCs are reproduced using Z-Sizer. The results can be seen in Table 3.

Sample Details
Sample Name: NLE NFT 80 mg en 3 mL3
SOP Name: manseltings.dat
General Notes:

| | |
|---|---|
| File Name: Mta NLE Nifurtimox_dts | Dispersant Name: Water |
| Record Number: 40 | Dispersant RI: 1.330 |
| Material RI: 1.4B | Viscosity (cP): 0.8870 |
| Material Absorbtion: 0.010 | Measurement Date and Time: |
| | Apr. 15, 2018 19:55:52 |

System

| | |
|---|---|
| Temperature (° C.): 25.0 | Duration Used(s): 50 |

Count Rate (Kcps): 330.5 Measurement Position (mm): 465
Cell Description: Disposable sizing cuvette Attenuator: 7

| | | | |
|---|---|---|---|
| Results | Size (d.nm): | % Intensity: | StDev(d.n |
| Z-Average(d.nm): 1470 | Peak 1: 50.03 | 70.6 | 13.92 |
| Pdl: 1.000 | Peak 2: 5.560 | 17.1 | 0.000 |
| Intercept: 0.153 | Peak 3: 1.704 | 12.3 | 0.4851 |

Result quality: Refer to quality report

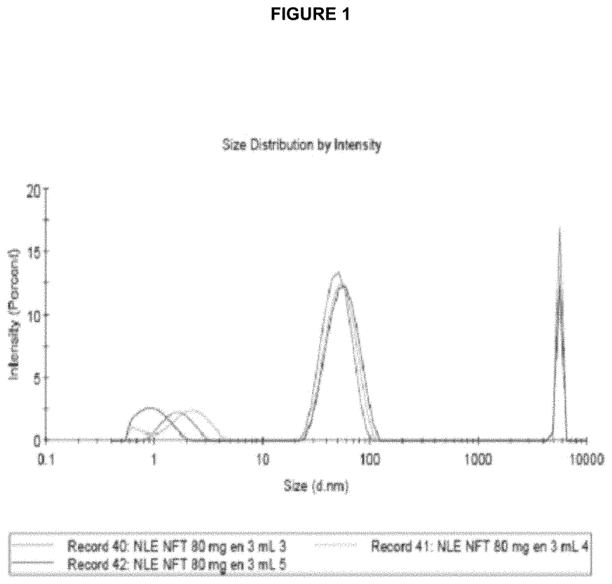

In turn, FIG. 1 shows that nifurtimox-carrying NLCs have a trimodal distribution to the average diameters of 1.7 nm, 50 nm and 5580 nm (the latter being obviously non-nanometric). However, by subjecting the suspension to a filter through a teflon filter of 220 nm, the measurement becomes bimodal, with an average diameter distribution of 1.0 nm (very predominant) with standard deviation of 0.05 nm, and a small part to the average diameter of 50 nm and standard deviation of 14.0 nm, having completely disappeared the mode of 5.6 microns.

In the filter, meanwhile, there is no yellowish remaining, no macroscopic lumps, circulating freely and effortlessly the washing water. The interpretation of this behavior is as follows: the particle size is 1 nm and the peaks of 50 and 5500 nm are produced by agglomerations thereof (probably thermodynamically generated due to the high surface/volume reason of the nano-objects), which are crumbled by the mechanical effect of filtering. As confirmation of this hypothesis, if the filtered liquid is allowed to rest for 48 hours, agglomerates of approximately 50 and 5000 nm spontaneously re-form, albeit in less quantity than in the original unfiltered suspension. Another confirmation lies in the direct observation of isolated NLCs (with a diameter of approximately 1 nm) and their agglomerates, through transmission electron microscopy and atomic force, the images of which are offered below.

TABLE 4
Comparative solubility and suspensibility It is observed
that the NLCs prepared by the inventors of the present invention
of nifurtimox, are very suspensible in water, compared to the
nifurtimox drug, favoring, as explained, the realization of
pharmacotechnical preparations.

| | distilled | absolute | Dimethyl | | |
|---|---|---|---|---|---|
| Product | water | ethanol | sulfoxide | Acetone | Chloroform |
| Nifurtimox | Insoluble | Soluble | very soluble | very | little |
| own | | | | soluble | soluble |
| Nifurtimox | Insoluble | Soluble | very soluble | very | little |
| Sigma-Aldrich | | | | soluble | soluble |
| Nanostructured | very | little | Soluble | very | little |
| Nanostructured | suspensible | soluble | | soluble | soluble |
| Lipid | | | | | |
| Nanoparticles | | | | | |
| NLC | | | | | |

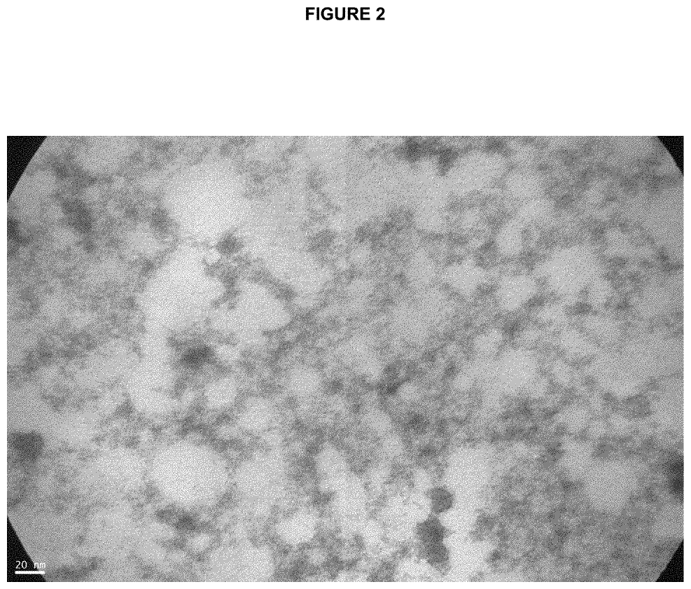

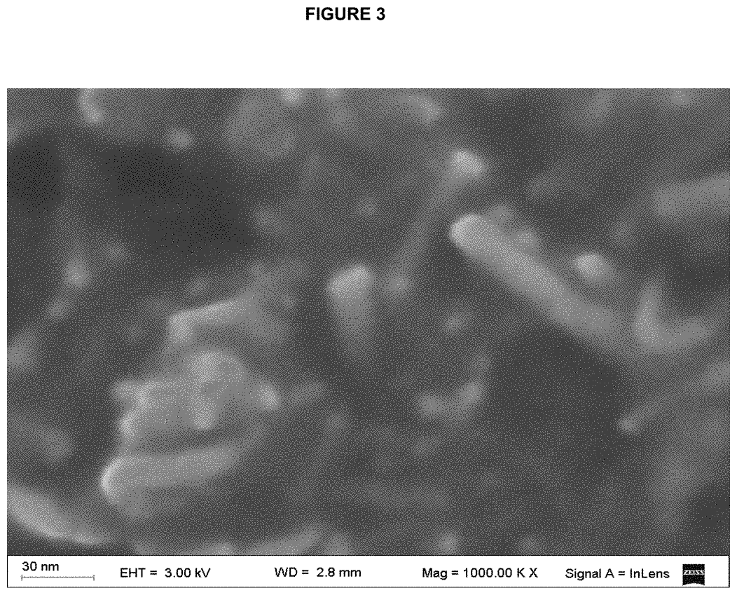

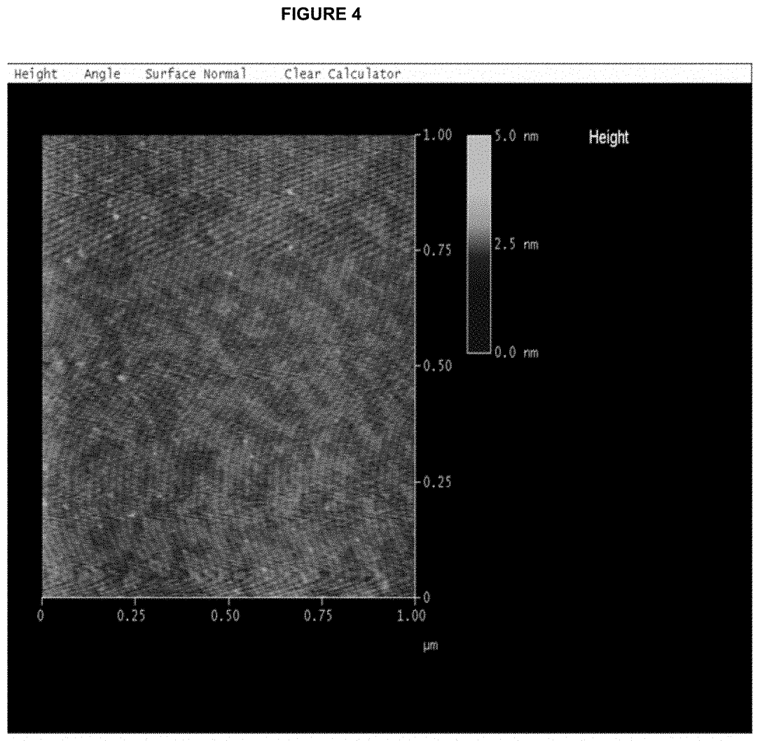

NLC Photomicrography:
1. Photography A) transmission electron microscope (TEM). Structured nanoparticles loaded with nifurtimox. Electronic transmission microscopy (TEM) performed in the First Chair of Histology of the Faculty of Medicine of the UBA on Oct. 29, 2012, with a contrast obtained with a solution of Osmium Tetroxide at 0.1% in a phosphate buffer, according to an ad hoc developed technique. Extension 400,000×. Nanoparticles measure approximately 1 nm and can be displayed as very abundant small rounded objects (SEE FIG. 2).
2. Photograph b) scanning electron microscope (BEM) Scanning Electron Microscopy (BEM) carried out at the Faculty of Exact Sciences of the University of Buenos Aires in 2016. Cylindrical NLC agglomerates approximately 20 nm in diameter may be observed. The resolution of the technique prevents separating the individual nanoparticles. (SEE FIG. 3).
3. Photography C) atomic force microscope (AFM). Atomic Force Microscopy showing the height of the NLCs prepared in the CIN-Nanof. It is observed that the particles are homogeneous, spheriform and appear to be about 2 nm tall. Laboratory of Microscopy of the Faculty of Exact Sciences of the University of Buenos Aires in 2016. (SEE FIG. 4)

### 4) EXAMPLES OF REALIZATION

The conventional method for NLC preparation is high pressure homogenization (HPH). THE preparation of NLC with HPH includes several critical process conditions, such as high temperature, high pressure and high concentration of surfactant. The high concentration of the surfactant turns out to be particularly problematic^{[32].} That is why it was decided to use the method of preparation of the “solvent diffusion”.

Removing Nifurtimox (NFX) from Tablets and Spectrophotometric Identification

NFX tablets with the fantasy name “Lampit”, prepared in the Republic of El Salvador and registered in the Republic of Guatemala, batch 09060093, with an expiration date in June 2014, containing 120 mg each of NFX and an average weight of 400 mg, were sprayed and subjected to the extraction of the active substance, through immersion in 10 ml of acetone per tablet, filtering and drying at 60° C. in porcelain crystallizer, repeating the operation with absolute ethanol. A powder consisting of yellow, acicular crystals, practically insoluble in water was obtained, with a yield of approximately 1 gram per 10 tablets. The powder mentioned contained an average 98.7% nifurtimox, in a spectrophotometric measurement against a primary Sigma-Ehrlich NFX standard at 401 nm wavelength.

Preparation of Nanoparticles

Following the solvent diffusion technique^{[32]} 10 mg of NFX was dissolved in a mixture in equal proportions of absolute ethanol and acetone, adding palmitic acid (PA) and linoleic acid (LA) to form a mixture 30/70% of both lipids. Each sample was heated to Mary's bath at 70° C. until full dissolution, finally spreading under mechanical agitation in 120 ml of water at 70° C. Then half of the samples were sonicated for 15 minutes. Once obtained, the nanoparticles were floculated by addition of hydrochloric acid 1 N up to pH 1.2 (about 13 ml per sample).

Treatment of the Aqueous Phase

The flocculated mixture, intense yellow and some turbidity, was filtered by medium grain paper, obtaining a clear liquid, yellow, pH 1.2, stable at room temperature. Subjected to spectrophotometric measurement it turned out to contain almost all of the NFX introduced into the system. Taken to neutrality with drops of 1N baking soda, it was desiccated into porcelain crystallizer at 40° C. for 48 hours. An amorphous, yellowish-brown powder was obtained and instantly re-suspended in the presence of distilled water, generating a clear, non-opalescent, intensely yellow liquid that absorbs light at 401 nm wavelength. There were no differences between sonicated and un sonicated samples.

Z-Sizer Suspension Study: Z Potential and Average Diameter

The macroscopically stable and clear liquid, filtered by 220 nm diameter pore teflon sieve, was subjected, using the Z-sizer, to the Z-potential examination, which turned out to be +8 mV. In the measurement of the average size of the suspended particles, a marked unimodal distribution of them was obtained in the approximate size of 0.95 nanometers. There were no differences between sonicated and un sonicated samples.

Examination of Particles by Transmission Electron Microscopy (TEM)

Images obtained by TEM with an magnification of 400,000×, show homogeneous particles, of about 1 nanometer, approximately spherical. The contrast was obtained with a 0.1% Osmium Tetroxide solution in a phosphate buffer, adapted ad hoc technique. (Examination carried out in the First Chair of Histology, Faculty of Medicine, University of Buenos Aires). There were no differences between sonicated and un sonicated samples.

Examination of Particles by Atomic Force Microscopy

The images obtained show spheroid particles approximately 1 to 2 nm in diameter, isolated or arranged in prisms approximately 200 nm thick and varied lengths. There were no differences between sonicated and un sonicated samples.

NFX Titration Present in Suspension and Particles

The NFX present in the colloidal suspension and particles was titled^{[37]} dissolving in acetone a desiccated sample of the suspension and recording the absorbance at 401 nm in spectrophotometer, against a witness prepared with the primary Sigma-Ehrlich pattern, obtaining a concentration of 0.09 mg/ml nifurtimox in colloidal suspension and approximately 4.7% p/p in desicced particles. There were no differences between sonicated and un sonicated samples.

Preparing a Concentrated NFX Suspension

In order to maintain experimental conditions, the initial stage of the preparation of the concentrated suspension repeated the technique described above using quantities 30 times greater than all reagents. The suspension at pH 1.2 was filtered directly through medium grain paper, neutralized with sodium bicarbonate 1 N, dried at 40° C. and dispersed again in distilled water at a concentration of 6 mg/ml of NFX, confirmed spectrophotometrically as described above.

Preparation of the Drinkable Concentrated Suspension

83.3 ml of the suspension obtained was available, 10 ml glycerin, 0.6 g hydroxyethyl cellulose 10000 Cp, 3 ml propylene glycol, 0.04 gr of succharin, 0, 125 g of methylparaben, 0.2 ml of vanilla essence, 1 ml of solution at 1% of sunset yellow dye and 2.5 ml of distilled water. In an erlenmeyer of adequate capacity the methylparaben and propylene glycol were placed and heated to full dissolution. In another container glycerin was incorporated and hydroxyethylcellulose dispersed, mixing until a homogeneous dispersion was obtained, with total absence of lumps; then the essence and dye were added to this dispersion, resuming this viscous preparation for the final mixture. Then, in a stainless steel pot, the entire 6 mg/ml NFX colloidal dispersion was introduced and the methylparaben/propylene glycol solution was added, constantly stirring to homogeneity. The sacarine was dissolved in all distilled water and incorporated into this homogeneous mixture. Finally, hydroxyethylcellulose dispersion was added in glycerin, essence and dye, and the assembly was processed for half an hour in a colloidal mill.

Title of Concentrated Drinkable Suspension

In a decanting ampoule, 8 ml of the drinkable concentrated suspension prepared with 12 ml of acetone was shaken, resulting in the extraction of NFX which was then titled as described in 1.6. The concentration obtained was 4.8 mg/ml of the active substance.

Description and Stability

The suspension obtained is homogeneous, clear, slightly opalescent, with a sirupous consistency, vanilla aroma, sweet taste and intense yellow color. It has remained macroscopically stable for one year since its preparation, both at room temperature and refrigerated at 4° C.

Preparation of NLC with Different Fatty Acids

All NLCs were prepared by the technique exposed, generating in all cases, an aqueous colloidal suspension of nanoparticles, with the concentration of 10 mg NFX (drug at 98% purity) per 100 ml of water (i.e. 10% mg/ml of NFX equivalent).

Fatty Acids Used

The following fatty acids were used:

Solid lipids at room temperature (between 19 degrees Celsius and 21 degrees Celsius, preferably at 20 degrees Celsius):
Stearic acid
Palmitic acid

Liquid lipids at room temperature (between 19 degrees Celsius and 21 degrees Celsius, preferably at 20 degrees Celsius):
Oleic acid
Linoleic acid
Palmitoleic acid
Arachydonic acid

The following preparations of nifurtimox-carrying nanoparticles were performed, combining solid fatty acids with liquid fatty acids in all cases:
Stearic/Oleic
Stearic/Linoleic
Stearic/Palmitoleic
Stebaric/Arachydonic
Palmitic/oleic
Palmitic/Linoleic
Palmitic/palmitoleic
Palmitic/arachydonic

Result of Preparations

In all cases yellow suspensions without turbidity that were carried at pH approximately 6.0 with NaOH 1 N and then filtered by Teflon filter of 220 nanometers in pore diameter.

Measuring the Zeta Potential of Particles

This indicator measures the attraction or repulsion between particles. In principle, the presence of a positive or negative load greater than 30 millivolts generates a strong repulsion between the particles and this stabilizes the suspension. If the load is lower or no, close to the isoelectric point, it is not supposed to favor the stability of the suspension. However, a potential Z greater than approximately 30 mV is not the only stability factor. There are other variables that influence the potential stability of the suspension, especially the size of the particles. A large size generates instability, so a small granulometry tends to stabilize the suspension. The end result depends on the balance between the independent variables.

TABLE 5
Measurement results: As checked, the eight samples
exhibit much lower electrical potential than (+ or −) 30 mV which
is considered a stability indicator. The one that moves further
away from the isoelectric point (0.0 mV) is the carrier of
stearic/linoleic acids, which however, with +16.5 mV is still
well below the limit that would indicate a predisposition to
stability from the electrical charge. Conclusions: (a) the Z-
potential of the particles studied cannot be considered as an
exclusive criterion of choice for any of the samples. (b) The
absence of turbidity for a period of one year and the macroscopic
stability of suspensions cannot be attributed to high Z
potential, an explanatory hypothesis being the extremely small
nature of the nifurtimox-laden NLCs under consideration.

| | POTENTIAL |
|---|---|
| COMBINATION OF FATTY ACIDS | “Z” |
| STERIC/OLEIC ACIDS | (+) 8.0 |
| STERIC/LINOLEIC ACIDS | (+) 16.5 |
| STERIC/PALMITOLEIC ACIDS | (+) 5.0 |
| STEBARIC/ARACHYDONIC ACIDS | (+) 3.0 |
| PALMITIC/OLEIC ACIDS | (+) 1.4 |
| PALMITIC/LINOLEIC ACIDS | (−) 0.2 |
| PALMITIC/PALMITOLEIC ACIDS | (+) 5.1 |
| PALMITIC/ARAQUIDONIC ACIDS | (+) 0.9 |

Determination of Size

Granulometry can be performed by volume criterion % and/or by light intensity %.

TABLE 6
Volume Size %. Very low granulometry is observed first
and foremost, ranging from 0.69 to 3.14 nanometers, which is
unusual in these nanoparticles that in other studies frequently
exhibit more than 100 nm of average diameter. Another aspect is
the unimodal character of the distribution since only a single
peak is observed for each type of combination of fatty acids,
and that peak concentrates 100% of the population. Examination
of the graphical representation of Volume % versus size in nm
(not shown here), shows that in the combination of Palmitic/Oleic
acids ONLY, of which 4 successive determinations were made, a
dispersive bias is observed in the results that removes precision
from the data of 3.14 nanometers, since some measurements give
peaks of approximately 1 and 8 nm. In the rest of the observations
the readings for each combination of fatty acids, were performed
from 4 to 6 repetitions and showed high accuracy.

| COMBINATION OF FATTY ACIDS | Nanometers |
|---|---|
| STERIC/OLEIC ACIDS | 0.91 |
| STERIC/LINOLEIC ACIDS | 0.72 |
| STERIC/PALMITOLEIC ACIDS | 3.01 |
| STEBARIC/ARACHIDONIC ACID | 1.3 |
| PALMITIC/OLEIC ACIDS | 3.14 |
| PALMITIC/LINOLEIC ACIDS | 0.69 |
| PALMITIC/PALMITOLEIC ACIDS | 0.73 |
| PALMITIC/ARAQUIDONIC ACIDS | 1.5 |

This modality is considered to be more significant than That of Volume % and offers more information.

TABLE 7
Granulometric study by % light intensity.
It is observed that the size distribution is polymodal. In the case of the
stebaric/oleic combination almost 60% of the granulometry
exceeds 10000 nanometers. In the case of the stephed/arachidonic
combination almost 80% of the granulometry exceeds 5000
nanometers. In the palmitic/arachydonic combination almost 70%
of the granulometry exceeds 7500 nanometers. These particulate
segments are obviously not nanometric. In the stebaric/linoleic
combination almost 70% of the particles exceed 300 nanometers.
In the palmitic/palmitoleic combination, more than 60% of the
particles exceed 400 nanometers. Since all suspensions had been
filtered by pore less than 220 nm 24 hours prior to measurement,
it is concluded that within a few hours the particles of the five
suspensions mentioned suffered strong bindings of diameter
greater than the pore mentioned, a process that, if continued
over time, could destabilize the suspension.

| | 1st | 1st | 2nd | 2nd | 3rd | 3rd | |
|---|---|---|---|---|---|---|---|
| COMBINATION OF | peak, | peak, | peak, | peak, | peak, | peak, | Total |
| FATTY ACIDS | nm | % | nm | % | nm | % | % |
| STERIC/OLEIC ACIDS | 0.91 | 40.1 | 0 | 0 | >10000 | 59.9 | 100 |
| STERIC/LINOLEIC | 0.77 | 17.1 | 5.57 | 14.3 | 307.8 | 68.6 | 100 |
| ACIDS | | | | | | | |
| STERIC/PALMITOLEIC | 3.26 | 19.5 | 147.5 | 80.5 | 0 | 0 | 100 |
| ACIDS | | | | | | | |
| STEBARIC/ARACHYDONIC | 0.95 | 20.8 | 0 | 0 | >5000 | 79.2 | 100 |
| ACIDS | | | | | | | |
| PALMITIC/OLEIC | 3.39 | 17.7 | 22.7 | 32.5 | 205.1 | 49.8 | 100 |
| ACIDS | | | | | | | |
| PALMITIC/LINOLEIC | 0.69 | 30.6 | 6.39 | 16.2 | 183.3 | 53.2 | 100 |
| ACIDS | | | | | | | |
| PALMITIC/PALMITOLEIC | 0.74 | 16.7 | 202.4 | 23.2 | 422.3 | 60.1 | 100 |
| ACIDS | | | | | | | |
| PALMITIC/ARACHYDONIC | 0.82 | 31.3 | 0 | 0 | >7500 | 68.7 | 100 |
| ACIDS | | | | | | | |

The distribution of the combination of esteric and palmitoleic acids shows a bimodal distribution, with a ratio of 20/80% of sizes of just over 3 and just over 147 nanometers. Since the volume % distribution offers in this combination a single peak of just over 3 nanometers, let us remember that with the low Z potential of +5 (which facilitates grouping), 147 nanometer particles are likely to constitute smaller particle bindings, facilitated thermodynamically. The polydispersion index (POI, not shown in the table, see below) is in this stepped/palmitoleic combination greater than 0.8 (stability is considered to improve below POI <0.5), which predisposes to the aforementioned hypothesis of weak agglutination around the peak of 147 nanometers.

The combination of palmitic/oleic acids is tritodal for the variable size/intensity %. Half of the nanoparticles are concentrated in a granulometry of 205 nanometers. Almost a third have an average diameter of 23 nanometers. Just under one-fifth (18%) it shows a size of just over 3 nanometers, practically identical to the data obtained with the “Volume %” method. The polydispersion index (POI) of this combination is 0.6 (not shown in the table, see below), higher than the POI index of 0.5 that is considered the stability limit, as stated above. Therefore, the hypothesis of the agglutination of minor particles around two thermodynamically facilitated grouping sizes of approximately 23 and 205 nanometers can also be used here.

Finally, the combination of palmitic/linoleic acids also offers a trimodal configuration of sizes. More than half of the particles are grouped into the 183 nanometers; almost a third of objects are less than 0.7 nanometers (same as determined by the volume method %) and the rest of the particles have just over 6 nanometers. A Polydispersion Index (POI) greater than 0.9 (not shown in the table, see below) induces us to think, even more so than in the previous two cases, that the nanoparticles of the two major peaks are affected by a tendency to clump. This is facilitated by the near-zero Z potential of −0.23 (see below), which facilitates this process as it does not in any way get in the way of the adhesion of nanoparticles to each other.

TABLE 8
Polydispersion Index (POI) for Light Intensity %

| COMBINATION OF FATTY ACIDS | Pdi |
|---|---|
| STERIC/OLEIC ACIDS | 0.9 |
| STERIC/LINOLEIC ACIDS | 0.7 |
| STERIC/PALMITOLEIC ACIDS | 0.8 |
| STEBARIC/ARACHYDONIC ACIDS | 0.8 |
| PALMITIC/OLEIC ACIDS | 0.6 |
| PALMITIC/LINOLEIC ACIDS | 1 |
| PALMITIC/PALMITOLEIC ACIDS | 0.8 |
| PALMITIC/ARACHYDONIC ACIDS | 0.9 |

Functional Effectiveness of Nifurtimox Nanoparticles in the In Vitro Treatment of Chagas Disease

Very significant differences in antiparasitic effectiveness between nifurtimox as such and nifurtimox were observed in nanoparticles nanostructured in in vitro models. Nifurtimox nanoparticles have a trypanicidal effect on trypomastigotes and amastigotes The use of Nifurtimox nanostructured nanoparticles has been almost two orders of magnitude more potent than Nifurtimox as such on trypanomas and three orders of magnitude more potent than Nifurtimox as such on kneading.

Blood trypomastigote Trials

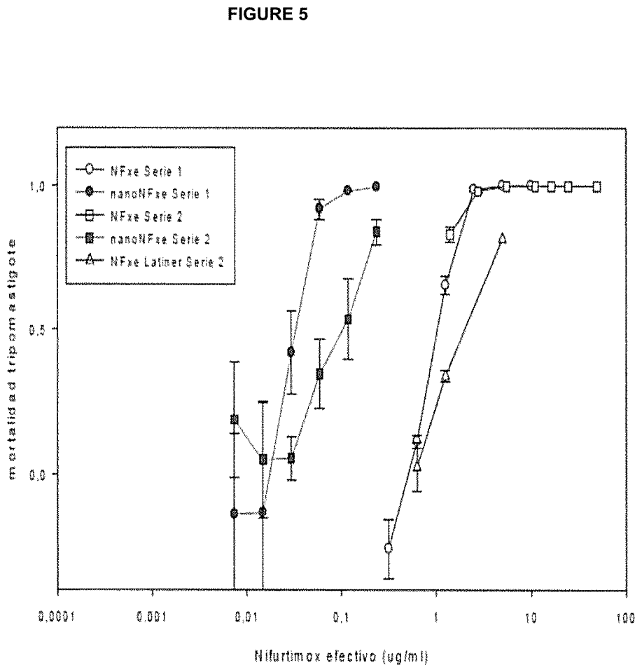

Blood trypomastigotes of the RA strain were purified from mouse blood during the parasitemia peak. The maintenance of the strain is usually carried out by successive weekly passages in CF1 mice, males of 21 days, infected with 10⁵ trypomastigotes via IP.
Triomastigote purification: Added 5 volumes of PBS 3% bovine fetal serum to blood obtained from infected mice. Erythrocytes were separated by low-speed sedimentation and incubated for 1 hour at 37° C. Finally the overnatant was centrifuged at 10,000×g for 30 minutes in order to obtain a high concentration of blood trypomastigotes for in vitro assays
Tryptonecidal Drug Effect: Incubation of 2.4×10⁵ trypomastigotes/well (100 L) in RPMI 5% SFB per triplicate in 96 wells plates with 100 L of the different concentrations of nifurtimox and nifurtimox nanoparticles in a final range of 0.3125 to 10 g/ml for 24 hrs to 37° C. and 5% CO₂.
Measurement of tripanocidal activity: Neubauer's chamber count considering 100% feasibility at the average count obtained for drug-free controls
   From the analysis of the figures below it emerges that doses two to three times smaller doses of nifurtimox nanoparticles were needed to reach 50% lethality (0.57±0.01 vs 1.45±0.46).
Nifurtimox nanoparticles on trypomastigotes:
   Dosage curves response to nifurtimox drug as such and nifurtimox nanoparticles on trypomastigote survival are shown (SEE FIG. 5).

Essays on Amastigotes

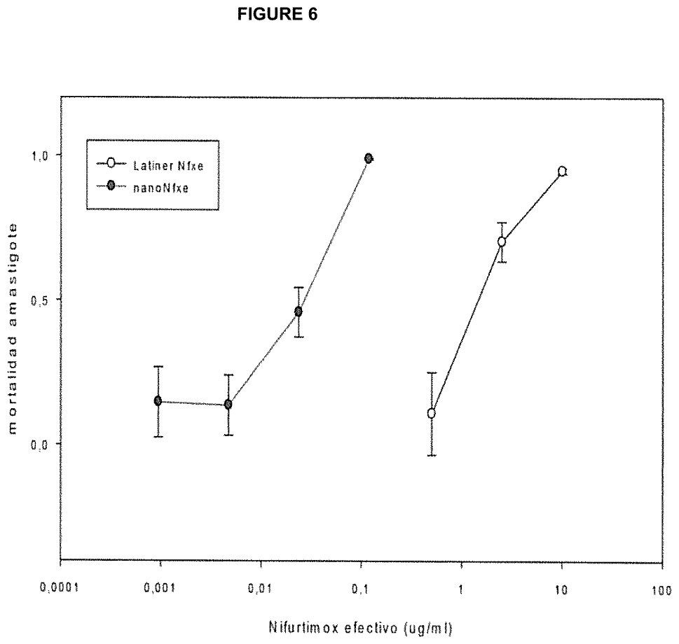

In vitro effectiveness of nifurtimox nanoparticles on central nervous system tumor cell apoptosis: astrocytomas and retinoblastomas (SEE FIG. 6).
Effect of nifurtimox nanoparticles on the viability of astrocytoma cells in cultivation. The cytocidal effect of nifurtimox nanoparticles on cultured Astrocytoma cells can be observed. It is interesting to note that GL26 NT cell lines are rat neural tumor, while spherical and adherent MSP12 lines, spherical MGG8 and adherents are human in cultivation. Inhibitory activity can be observed in all exposed cells from 500 mM (SEE FIG. 6).

Cell infection: 5×104 Vero/well cells were sown on glass laminillas on 24 wells plates for 24 hrs to 37 C and 5% CO₂. They were then incubated with blood trypomastigotes of the RA strain at the rate of 5 parasites/cell (MOI: 5) for 3 hrs.

Effect of nanoparticles with Nftx on T kneadings. cruzi: Completed infection, the cells were washed in order to eliminate free and incubated parasites with Nftx (0.02-10 g/ml) and nano Nftx (0.02-2.5 g/ml) by triplicate for 72 hrs at 37° C. and 5% CO₂. They were finally fixed with methanol for 5 minutes and dyed with Giemsa. In order to measure parasitic load per cell, random photos of 20 infected cells were taken for each drug concentration tested.

The number of kneads/cell by image analysis was determined using Image J software.

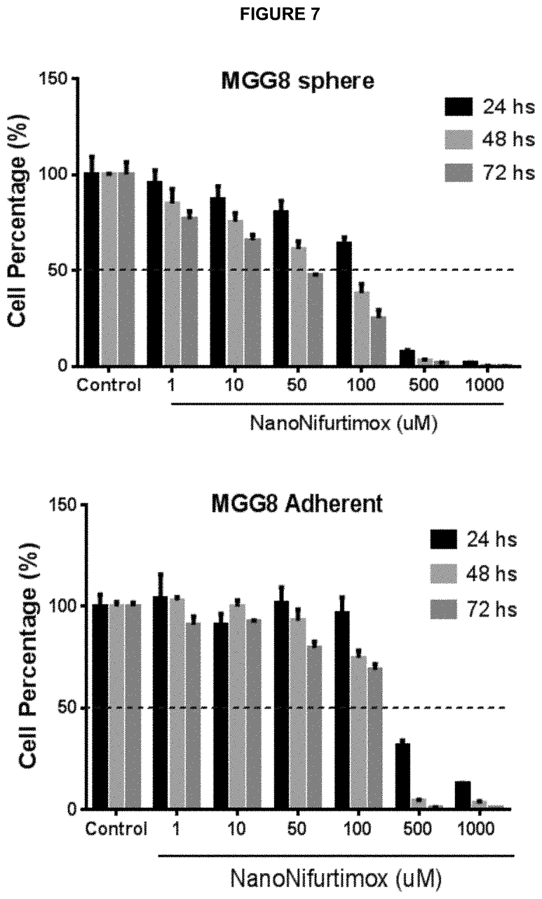

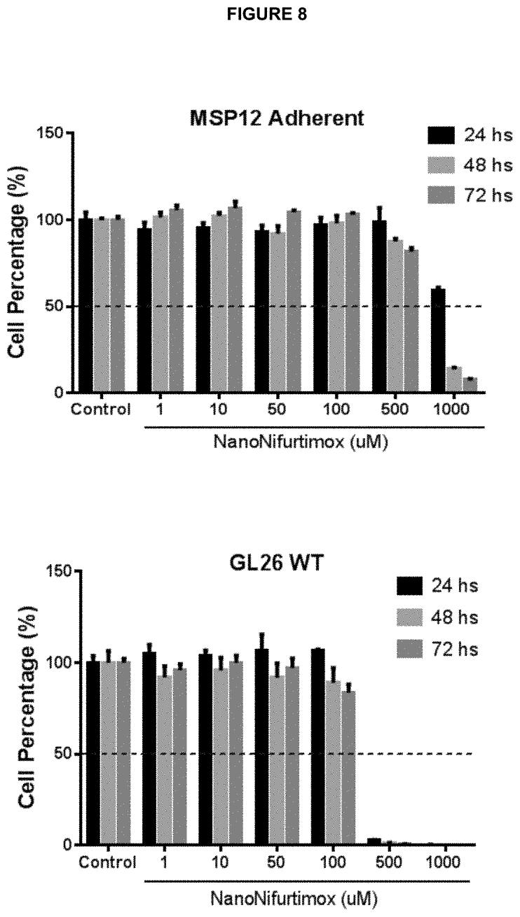

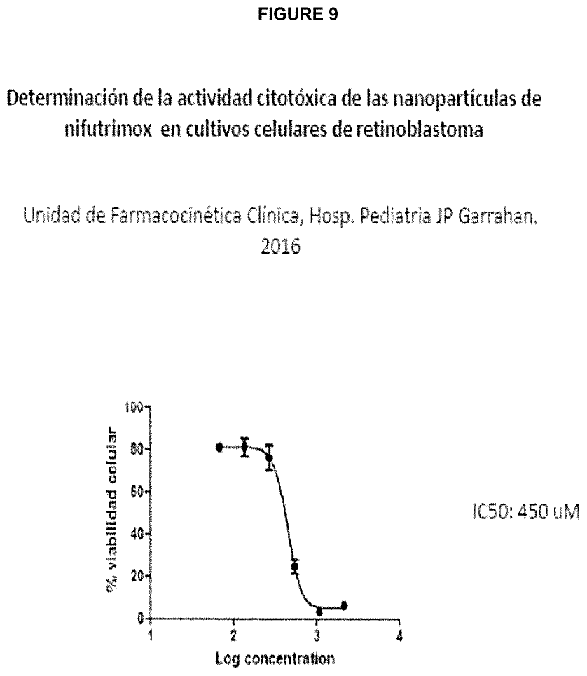

We consider that the process of nanoparticulation of nifurtimox, allows an increase of transmembrane passage and an increased intracellular bioavailability of the drug, which gives the nifurtimox a new and fundamental quality for therapeutic objectives, namely: high efficacy on intracellular kneading nests which could eventually become an effective therapeutic for chagas disease in chronic phase, today intractable.
Nifurtimox nanoparticles on amastigotes:
   Dosage curves response to nifurtimox drug as such and nifurtimox nanoparticles on the survival of amastigotes are shown (SEE FIGS. 7 and 8).
   FIG. 9 shows the effect of nifurtimox nanoparticles on the viability of retinoblastoma cells in cultivation. In vitro antineoplastic activity of nifurtimox nanoparticles on cells in Retinoblastoma culture is observed. The cytotoxicity test was conducted on commercial cell lines of retinoblastoma Y79. They were sown at the rate of 20,000 cells per well in 96-well plates. Cell proliferation was measured by the MTT colorimetric method.
   Also, the inventors emphasize that the composition of the invention has the following relevant characteristics that deserve to be commented on, they are:
Its administration is oral, pulmonary, intravenous, rectal, ophthalmic, colonic, parenteral, intracisteral, intrathecal, intravaginal, intraperitoneal, ótica, local, transdermal, oral, nasal and topical.
Transdermal penetration enhancers (enhancers) of the sulfoxide family are used especially so-called dimethylsufoxide (DMSO), dimethylacetamide (DMAC) and dimethylformamide (DMF), in the urese oxazolidone family, of azones and pyrrolidones, the family of essential oils such as terpenes and terpenoids and the family of fatty acids
The dosage form is selected from liquid dispersions, gels, aerosols, ointments, creams, freeze-dried shapes, tablets, orodispersible tablets, capsules, colloidal suspensions, removable papers and jet (needle free).
The dosage form is selected from or combinations of immediate release, controlled release, delayed release, long release, throbbing release formulations.
It comprises one or more excipients, vehicles or combination of the same pharmaceutically stable.
The additional active substance is selected from the group of diuretics, α-blockers, β-blockers, ACE inhibitors, calcium channel blockers, angiotensin antagonist, nervous system inhibitors and vasodilators.

### Bibliographic References Cited in the Descriptive Memory

[1] Pan American Health Organization (PAHO). Quantitative estimation of Chagas disease in the Americas. Montevideo, PAHO/HDM/CD/425, 2006.
[2] J. C. Rodrigues Coura and S. L. Castro. “A Critical Review on Chagas Disease Chemoterapy”. Memoirs of the Oswaldo Cruzlnstitute, vol. 97, no. 1, pp. 3-24, 2002.
[3] A. M. Strosberg, K. Neighborhood, V. H. Stinger, J. Tashker, J. C. Wilbur, L. Wilson, and K. Woo. Chagas disease: a Latin American nemesis, 2007.
[4] C. M. Morel. “Reaching maturity-25 years of the TDR”. Parasitology Today, vol. 16, pp. 522-525, 2000.
[5] A. Moncayo. “Chagas disease: current epidemiological trends after the interruption of vectorial and transfusional transmission in the Southern Cone countries”. Memoirs of the Oswaldo Cruzlnstitute, vol. 98, pp. 577-591, 2003.
[6] D. A. Leiby, J. R. M. Herron, E. J. Read, B. A. Lenes, and R. J. Stumpf. “Trypanosoma cruzi in Los Angeles and Miami blood donors: impact of evolving donor demographics on seroprevalence and implications for transfusion transmission”. Transfussion, vol. 42, pp. 549-55, 2002.
[7] C. B. Beard, G. Pye, F. J. Steurer, R. Rodriguez, R. Campman, and A. T. Peterson. “Chagas disease in a domestic transmission cycle, southern Texas, USA”. Emergency Infection Disease, vol. 9, pp. 103-105, 2003.
[8] J. H. Diaz. “Recognizing and reducing the risks of Chagas disease (American trypanosomiasis) in travelers”. Journal Traveller Medicine, vol. 15, pp. 184-195, 2008.
[9] A. Prata. “Clinical and epidemiological aspects of Chagas' disease”. Lancet Infection Disease, vol. 2, pp. 92-100, 2001.
[10] J. A. Urbina and R. Docampo. “Specific Chemoterapy of Chagas disease: controversies and advances”. Trends Parasitology, vol. 93, pp. 203-214, 2003.
[11] G. C. Levi, V. Amato, J. F. Net, and D. Araujo. “Analise de manifestacoes colaterais devidas ao use of medicine Ro-7-1051 nitromidazolic specific da doenca de Chagas”. Journal of the Institute of Tropical Medicine of Sao Paulo, vol. 17, pp. 49-52, 1975.
[12] A. L. de Andrade, F. Zicker, R. M. Oliveira, S. Almeida Silva, A. Luquetti, L. R. Travassos, I. C. Almeida, S. S. of Andrade, J. G. Andrade, and C. M. Martinelli. “Randomised trial of efficacy of benznidazole in treatment of early Trypanosoma cruzi infection”. Lancet, vol. 348, pp. 1407-1413, 1996.
[13] S. Sosa Estani, E. L. Safe, A. M. Ruiz, E. Velazquez, B. M. Porcel, and C. Yamporis. “Efficacy of chemotherapy with benznidazole in children in the indeterminate phase of Chagas disease”. American Journal of Tropical Medical Hygiene, Vol. 59, pp. 526-529, 1998.
[14] J. R. Cancado. Criteria of Chagas disease cure. Memoirs of the Oswaldo Cruz Institute, vol. 94, pp. 331-336, 1999.
[15] S. Sosa Estani and E. L. I'm sure. “Treatment of Trypanosoma cruzi infection in the undeterminated phase. Experience and current guidelines in Argentina”. Memoirs of the Oswaldo Cruz Institute, vol. 94, pp. 363-365, 1999.
[16] S. Murta, R. Gazzinelli, Z. Brener, and A. Romanha. “Molecular characterization of susceptible and naturally resistant strains of Trypanosoma cruzi to benznidazole and nifurtimox”. Molecular Biochemical Parasitology, vol. 93, pp. 203-214, 1998.
[17] C. Breyrer, J. C. Villar, V. Suwanvanichkij, S. Singh, S. Baral, and E. J. Mills. “Neglected diseases, civil conflicts, and the right to health”. Lancet, vol. 370 pp. 619-627, 2006.
[18] Fr. Trouiller, P. Olliaro, E. Torreele, J. Orbinski, R. Laing, N. Ford. “Drug development for neglected diseases: a deficient market and a public-health policy failure”. Lancet vol. 359, pp. 2188-2194, 2002.
[19] M. S. Braga, L. Lauria-Pires, E. R. Arganaraz, R. J. Nascimento, and A. R. L. Texeira. Journal of the Institute of Tropical Medicine of Sao Paulo, vol. 42, no. 3, May/June, 2000.
[20] R. do Campo and S. N. J. Moreno. Fed. Proceedings, vol. 45, pp. 2471-2476, 1986.
[21] WHO/Make medicines child size. Available at: http://www.who.int/childmedicines/en (accessed July 2009. Ref. D. A. Chiapetta, C. Hocht and A. Sosnik “A Highly Concentrated and Taste-Improved Aqueous Formulation of Efavirenz for a More Appropriate Pediatric Management of the Anti-HIV Therapy”, Current HIV Research, vol. 8, pp. 223-231, 2010.
[22] J. F. Standing and C. Tuleu. “Paediatric formulations-getting to the heart of the problem”. International Journal Pharmaceutical, vol. 300, pp. 56-66, 2005.
[23] I. Choonara, and S. Conroy. “Unlicencied and off-label drug use in children. Implications for safety”. Drug Safety, vol. 25, pp. 1-5, 2002.
[24] T. Nunn and J. Williams. “Formulations of medicines for children”. British Journal of Clinical Pharmacology, vol. 59, pp. 674-676, 2005.
[25] T. Eileen Kairuz, D. Gargiulo, C. Bunt, and S. Garg. “Quality, safety and efficacy in the “off-label” use of medicines”. Current Drug Safety, vol 2, pp. 89-95, 2007.
[26] E. L. Romero and M. J. Morilla, “Nanotechnological approaches against Chagas disease”. Advanced Drug Delivery Reviews, doi:10.1016/addr.2009.11.025, 2009.
[27] Fr. Severino, T. Andreani, A. S. Macedo, J. F. Fangueiro, M. H. A. Santana, A. M. Silva, and E. B. Souto. “Current State-of-Art and New Trends on Lipid Nanoparticles (SLN and NLC) for Oral Drug delivery” Hindawi Publishing Corporation. Journal of Drug Delivery. Vol. 2012. Article ID 750891, 10 pages. Doi:10.1155/2012/750891, 2012.
[28] M. Radke, E. B. Souto and R. H. Muller. “Nanostructured Lipid Carriers: A Novel Generation of Solid Lipid Drug Carriers”. Pharmaceutical Technology Europe, vol. 17, no. 4, pp. 45-50, 2005.
[29] Y. Kawashima, H. Yamamoto, H. Takeuchi, T. Hino, and T. Niwa. “Properties of a peptide containing DL-lactide/glycolide copolymer nanospheres prepared by novel emulsion solvent diffusion method”. European Journal of Pharmaceutics and Biopharmaceutics, vol. 45, pp. 41-48, 1998.
[30] F. Q. Hu, H. Yuan, H. H. Zhang, and M. Fang. “Preparation of solid lipid nanoparticles with clobetasol propionate by a novel solvent diffusion method in aqueous system and physicochemical characterization”. International Journal of Pharmaceutics, vol. 239, pp. 121-128, 2002.
[31] F. Q. Hu, Y. Hong, and H. Yuan. “Preparation and characterization of solid lipid nanoparticles containing peptide”. International Journal of Pharmaceutics, vol. 273, pp. 29-35, 2004.
[32] F. Q. Hu, S. P. Jiang, Y. Z. Du, H. Yuan, Y. Q. Ye, and S. Zeng. Preparation and characterization of stearic acid nanostructured lipid carriers by solvent diffusion method in an aqueus system”. Coloids and Surfaces B: Biointerfaces. Vol 45, pp. 167-173, 2005.
[33] R. H. Muller, K. Mader, and S. Gohla. “Solid Lipid Nanoparticles (SLN) for controlled drug delivery—a review of the state of the art”. European Journal of Pharmaceutics and Biopharmaceutics, vol. 50, pp. 161-177, 2000.
[34] R. H. Muller, S. Maaben, H. Weyhers, F. Specht, and J. S. Lucks. “Cytotoxicity of magnetite loaded polylactide, polylactide/glycolide particles and solid lipid nanoparticles (SLN)”. International Journal of Pharmaceutics, vol. 138, pp. 85-94, 1996.
[35] R. H. Muller, D. Ruhl, and S. A. Runge. “Biodegradation of solid lipid nanoparticles as a function of lipase incubation time”. International Journal of Pharmaceutics, vol. 144, pp. 115-121, 1996.
[36] A. Moncayo. “Chagas disease: current epidemiological trends after the interruption of vectorial and transfusional transmission in the Southern Cone countries”. Memoirs of the Oswaldo Cruzlnstitute, vol. 98, pp. 577-591, 2003.
[37] Sanchez G, Cuellar D, Zulantay I, Gajardo M, Gonzalez-Martin G. Cytotoxicity and trypanocidal activity of nifurtimox encapsulated in ethylcyanoacrylate nanoparticles. Biological Research v. 35, n. 1, Santiago 2002.
[38] Saulnier Sholler G L, Brard L, Straub J A, Dorf L, Illyene S, Koto K, Kalkunte S, Bosenberg M, Ashikaga T, Nishi R. Nifurtimox induces apoptosis of neuroblastoma cells in vitro and in vivo. J Pediatric Hematol Oncol. 31 (3) pp 187-193. 2009.
[39] Stanchi K M, Bruchelt G, Handgretinger R, Holzer U. Nifurtimox reduces N-Myc expression and aerobic glycolysis in neuroblastoma. Cancer Biol Ther. 2015; 16(9):1353-63.

1) Pharmaceutical composition of antineoplastic and antitumor action, characterized by the production of structured lipid nanoparticles that are between 0.1 nm and 100 nm in diameter, spheroidal in shape and contain particles of one or more active substances for the treatment of diseases produced by trypanosomes and for the treatment of tumors of neural origin, and at least one coupling of two fatty acids consisting of a saturated fatty acid or semi-acid lipid at the temperature between 19° C. and 21° C. and an unsaturated fatty acid or liquid at the temperature between 19° C. and 21° C.

2) Pharmaceutical composition of antineoplastic and anti-tumor action, according to claim 1 wherein said coupling of two saturated and unsaturated acids have a training temperature preferably of 20° C.

3) Pharmaceutical composition, according to claim 1, wherein said active substance for trypanosomiasis is selected between benzimidazole, nifurtimox, ertanidazole, buthionine sulfoximine, derivatives of nitroimidazole, eflornitine, melarsoprol, pentamidine, surimin and fexinidazole.

4) Pharmaceutical composition, according to claim 3, wherein said active substance for trypanosomiasis is selected between benznidazole, nifurtimox or the combination of benznidazole nanoparticles and nifurtimox nanoparticles.

5) Pharmaceutical composition, according to claim 1, wherein saturated fatty acid or semi-solid lipid is selected between esteric acid and palmitic acid, and unsaturated fatty acid or liquid lipid is selected from oleic acid, arachydonic acid, palmitoleic acid, alpha-linoleic acid, or gamma-linoleic acid.

6) Pharmaceutical composition, according to claim 1, wherein said active substance is buthionine sulfoximine, for the treatment of tumors of neural origin preferably pediatric tumors of neural origin.

7) Pharmaceutical composition, according to claim 1, wherein said coupling of two fatty acids comprises the saturated fatty acid palmitic acid.

8) Pharmaceutical composition, according to claim 1, wherein said coupling of two fatty acids comprises the saturated fatty acid stearic acid.

9) Pharmaceutical composition, according to claim 1, wherein said coupling of two fatty acids comprises polyunsaturated fatty acid linoleic acid.

10) Pharmaceutical composition, according to claim 1, wherein said coupling of two fatty acids comprises unsaturated fatty acid palmitoleic acid.

11) Pharmaceutical composition according to claim 1, wherein these structured lipid nanoparticles are preferably sized from 1 nm to 10 nm in diameter.

12) Pharmaceutical composition according to claim 11, wherein such structured lipid nanoparticles have a size preferably of 1 nm.

13) The pharmaceutical composition according to claim 1, characterized because it is a medicine for the treatment of Chagas Mazza disease.

14) The pharmaceutical composition according to claim 1, characterized because it is a medicine for the treatment of sleep disease (human African trypanosomiasis) produced by the parasitic Trypanosoma brucei gambiense and transmitted by the vector (glossin fly)tse-tsé or for the treatment of skin or visceral leishmaniasis, which is a disease caused by infection of several species of Leishmania, a protozoan parasite in the family Trypanosomatidae or other diseases caused by protozoa such as Toxoplasmosis, malaria and trichomoniasis. Toxoplasma: family Sarcocystidae; Malaria (malaria): Plasmodiidae family; Trichomona: family Trichomonadidae.

15) The pharmaceutical composition, according to claim 1, characterized because it is a medicine for the treatment of tumor diseases of the central nervous system such as astrocytoma, retinoblastoma and neuroblastoma.

16) The pharmaceutical composition according to claim 1, characterized because it is a drug where the active substance is nifurtimox associated with an antitumor drug of the cytostatic, hormonal or immune type.

17) The pharmaceutical composition according to claim 1, wherein it is a medicine where preparations of a cytostatic, hormonal or immunological nature are administered at lower doses than those currently indicated.

18) The pharmaceutical composition according to claim 1, characterized because it is a medicine for the treatment of diseases and tumors called rare or orphans known as: ependymoma, neurofibromatosis type I and II, carcinoids, primitive neuroectodermal tumor, pheochromocytoma, neuroendochrine tumor, VIPoma

19) A method of obtaining such a pharmaceutical composition according to claim 1, characterized because it comprises the following steps:
a) Weighing 140 to 160 mg, preferably 150 mg of active substance for trypanosomiasis, 1300 mg solid saturated fatty acid at temperature from 19 degrees Celsius to 21 degrees Celsius, preferably at 20 degrees Celsius (which will be called “ambient temperature”), and 440 mg of liquid unsaturated fatty acid at temperatures of 19 degrees Celsius to 21 degrees Celsius, preferably at 20 degrees Celsius both pharmacopoeia quality;
b) Measure 55 ml to 65 ml, preferably 60 ml of analytical quality acetone, from 55 ml to 65 ml, preferably 60 ml of absolute ethyl alcohol of analytical quality;
c) Mix the phase b liquids in a Erlenmeyer glass flask with the 2000 ml borosilicate capacity, and heat between 60.0 degrees Celsius to 80.0 degrees Celsius, preferably to 70.0 degrees Celsius;
d) Place the flask with the mixture of heated phase c liquids in a heated magnetic stirrer, regulated at the same temperature, and add the active substance, solid saturated fatty acid at room temperature and liquid fatty acid at room temperature under agitation from 250 rpm to 300 rpm, preferably at 275 rpm, for 10 minutes to 20 minutes, preferably 15 minutes. A clear yellow liquid is obtained;
e) Without interrupting the stirring, add 1000 ml of preheated distilled water from 65° C. to 75° C., preferably at 70° C., keep stirring and heating at stipulated speed and temperature for 10 to 20 minutes, preferably 15 minutes and a bright yellow cloudy liquid is obtained,
f) Suspend heating and maintain agitation at the stipulated speed, 60 ml of Hydrochloric acid 1N is added and stirring is maintained for 15 minutes;
g) The preparation of stage f), let stand until the ambient temperature acquires and is filtered with fine grade filter paper, the liquid phase of the mixture offers a clear appearance of an intense yellow color and the precipitate obtained is un abundant, amorphous, white and the pH of the liquid is 2.0;
h) Place the obtained liquid in stage (g), again in agitation at room temperature and gradually add a NaOH 1N solution in 5 ml portions, after the incorporation of each serving, the pH must be controlled and the operation is suspended when the pH is 5.5;
i) Filter the mixture again by fine grain filter paper, the precipitate is very in abundant, white and the liquid is completely clear and intense yellow;
j) Filter the liquid through a Teflon filter with 220 nanometer pores and it is stored in clean sterile glass or PVC containers, in cool place (can be fridge at 4° C., but not freezer) and sheltered from light; and
k) Finally obtain the liquid containing the nanoparticles of one or more active substances for trypanosomiasis in colloidal suspension, the Z potential ranges from −30 to +30, preferably +8, the particles measured by Z-sizer and ultramicroscopy have from 0.1 nanometers to 100 nanometers, preferably 1 to 10 nanometers, particularly 1 nanometer in diameter and spheroidal shape.
